(19) United States
(12) Patent Application Publication
(10) Pub. No.: US 2015/0290271 A1
(43) Pub. Date: Oct. 15, 2015

(54) Nutraceutical Composition Obtained from Fungus-Challenged Soy Seedlings

(71) Applicants: Maria Anna Verbruggen, DH Renkum (NL); Ruud Simons, Ep Alphen aan den Rijn (NL); Holger Niessen, Thalwil (CH); Jean-Paul Vincken, DH Renkum (NL); Harm Gruppen, NA Wageningen (NL)

(72) Inventors: Maria Anna Verbruggen, DH Renkum (NL); Ruud Simons, Ep Alphen aan den Rijn (NL); Holger Niessen, Thalwil (CH); Jean-Paul Vincken, DH Renkum (NL); Harm Gruppen, NA Wageningen (NL)

(73) Assignee: Frutarom Switzerland Ltd., Wädenswil (CH)

(21) Appl. No.: 13/809,666

(22) PCT Filed: Jul. 5, 2011
(86) PCT No.: PCT/CH2011/000158

§ 371 Date: Jun. 14, 2013

(30) Foreign Application Priority Data
Jul. 12, 2010 (CH) 1133/10

(51) Int. Cl. A61K 36/48; A61K 31/37; A61K 31/7048; A01G 1/00; A61Q 19/00; A61Q 5/00; A61Q 3/00; A61K 31/352; A61K 8/97

(52) U.S. Cl. CPC A61K 36/48; A61K 31/352; A61K 31/37; A61K 31/7048; A61K 8/97; A61Q 19/00; A61Q 5/00; A61Q 3/00; A01G 1/001; A61K 2236/19

(57) ABSTRACT

Soybean seedlings and therefrom extractable compositions are described. Such compositions comprise prenylated isoflavones and at least one isoflavonoid, said isoflavonoid being selected from one of the chemical classes of isoflavones, coumestans and pterocarpans. Such compositions usually comprise at least 5% prenylated isoflavones, in particular prenylated isoflavones selected from prenylated daidzein, prenylated hydroxydaidzein, prenylated glycitein, prenylated hydroxygenistein, and prenylated genistein. Such compositions also comprise pterocarpans, preferably in amounts of at least 20%, and with a novel ratio of glyceollin I to glyceollin II to glyceollin III to glyceollin IV. Also described is a production method comprising the step of fermenting the soybeans under stress, in particular in the presence of cultures of fungi, preferably in the presence of Rhizopus microsporus Var. oryzae.

### CROSS REFERENCES TO RELATED APPLICATIONS

This application claims the priority of Swiss patent application 1133/10, filed Jul. 12, 2010, the disclosure of which is incorporated herein by reference in its entirety.

### TECHNICAL FIELD

This invention relates to the production of soy seedlings, in particular nutraceutically improved soy seedlings.

### BACKGROUND ART

There is an increasing interest to identify phytochemicals or plant compounds with health-promoting activities. In vitro screening assays to identify these bioactive compounds cover a broad area of research including anti-oxidant, anti-cancer, anti-obesity, cholesterol-lowering, receptor mediating and many other activities. Often, successful characterization of a phytochemical can lead to the development of new supplements with health-promoting activities. Supplements containing health-promoting activity are referred to as nutraceuticals. Plants produce a diverse array of over 100,000 low molecular weight natural products known as secondary metabolites (Boué et al. 2009). These secondary metabolites differ from the components of primary metabolism because they are generally considered not essential to basic plant metabolic processes. Most of these compounds are derived from various plant pathways, including the isoprenoid, phenylpropanoid, alkaloid, or fatty acid/polyketide pathways. One group of important secondary metabolites is the group of flavonoids. Flavonoids are ubiquitous in many plants and provide utility for the plant as flower pigments to attract pollinating insects, UV protectants, signal molecules to symbionts, and defence against pathogens. Isoflavonoids are a subclass of flavonoids and are the constitutive secondary metabolites found primarily in legumes. The subclass of isoflavonoids comprises sub-subclasses of which the isoflavones, coumestans and pterocarpans are relevant for the invention described. Table 1 represents this nomenclature.

TABLE 1
Nomenclature used:

| Class | Subclass | Sub-subclass |
|---|---|---|
| Flavonoids | Isoflavonoids | Isoflavones |
| | | Coumestans |
| | | Pterocarpans |
| | | . . . |

Important health-promoting activities have been linked to legume consumption, including reduced risk of various cancers and coronary heart disease (Boué et al. 2009; Mazur et al. 1998; Messina et al. 1998; Price et al. 1985). The best known legume to contain nutritionally relevant amounts of isoflavonoids is soybean. The isoflavone aglycones genistein, daidzein, and glycitein, along with their respective glucoside forms (genistin, malonyl genistin, acetyl genistin, daidzin, malonyl daidzin, acetyl daidzin, glycitin, malonyl glycitin and acetyl glycitin), are the predominant isoflavones in soybean. Many soy foods and supplements that are considered to be functional foods have high concentrations of the constitutive isoflavones daidzein and genistein. Also sprouts from legume sources, including soybean, are commonly consumed. In soybean sprouts one might find coumestrol, which can be formed from daidzein during sprouting.

Nowadays, also phytoalexins gain interest with nutritionists. Many phytoalexins can be chemically classified as flavonoids. Phytoalexins are low molecular weight compounds that are synthesized de novo and accumulate in plants in response to infection or stress due to wounding, freezing, ultraviolet light exposure, or exposure to microorganisms. Phytoalexin biosynthesis can be manipulated by application of abiotic (non-living) or biotic (living) factors that stress the plant into producing or releasing greater phytoalexin concentrations (Boué et al. 2009; Graham et al. 1991; Graham et al. 1990; Paxton 1991). Antifungal, antimicrobial, and antioxidant activities are some of the beneficial activities of phytoalexins that help to enhance the survival of the soybean plant or seedling during stress induction (Dakora et al. 1996). Phytoalexins have been well documented in the field of plant defence. Much research has been conducted on the elicitation process, and specific elicitors have been discovered. However, phytoalexins are only recently being explored as nutritional components and a source for development of health promoting food products. The glyceollins (I, II, and III) are the predominant soybean phytoalexins studied. They belong to the sub-subclass of pterocarpans, and show antimicrobial activity against numerous soybean pathogens. Recent research has shown that the glyceollins have anti-estrogenic and anticancer activities (Burow et al. 2001; Salvo et al. 2006; Wood et al. 2006). Further work has shown that extracts from elicitor-treated soybeans have higher antioxidant activities when compared to untreated controls (Feng et al. 2007). Glyceollin I, glyceollin II and glyceollin III are usually present in elicitated soybean seedlings in the ratio of 1 to 2 to 6 (Keen et al. 1986). Depending on the plant part other ratios may be found. Soy phytoalexins from the sub-subclass of pterocarpans, long known only as plant defensive antimicrobials, are now being viewed as beneficial plant compounds that can be considered alongside other soy isoflavonoids when health promoting properties are evaluated. Some of these phytoalexin compounds as well as isoflavonoid derivatives have been tested for their ability to bind to the estrogen receptors alpha and beta. In general such binding tests are done with HPLC purified components that are tested for relative binding to estrogen receptor alpha and beta using a variety of standard assays. Such assays result in an IC50, presenting the concentration at which 50% of the receptors are bound by the test compound. IC50 values have been published for the compounds indicated in the Table 2 below. For some compounds more than one 1050 have been reported. The exact values of the IC50s seem to depend on the details of the estrogen binding assay used (see Table 2).

TABLE 2

| | ER alpha | ER beta | |
|---|---|---|---|
| Compound | IC50 (M) | IC50 (M) | Reference |
| Daidzein | 3.2E−06 | 1.1E−06 | (Sun et al. |
| | | | 2008) |
| | 1.7E−05 | 1.2E−06 | (Booth et al. |
| | | | 2006) |
| Glycitein | 5.5E−06 | 1.1E−06 | (Sun et al. |
| | | | 2008) |
| Genistein | 8.40E−07 | 3.5E−08 | (Sun et al. |
| | | | 2008) |
| | 3.0E−07 | 2.0E−08 | (Booth et al. |
| | | | 2006) |
| Coumestrol | 3.0E−07 | 5.9E−09 | (Sun et al. |
| | | | 2008) |
| | 6.0E−08 | 2.0E−08 | (Booth et al. |
| | | | 2006) |
| | | 4.0E−08 | (Han et al. |
| | | | 2002) |
| Glyceollin I | 1.7E−06 | | (Zimmermann |
| | | | et al. 2010) |
| Glyceollin II | 6.6E−06 | | (Zimmermann |
| | | | et al. 2010) |
| Glyceollin | >10E−06 | | (Zimmermann |
| III | | | et al. 2010) |
| Glycinol | 1.4E−08 | 9.1E−09 | (Boué et al. |
| | | | 2009) |

It has further been described that prenylated genistein (Kretzschmar et al. 2010) and prenylated OH-genistein (Okamoto et al. 2006) act similarly towards estrogen receptor alpha as genistein. A study by Ahn et al. (2004) suggested that prenylated derivatives of genistein may act as antagonists.

The capability to bind to the estrogen receptors is interpreted as indicative for in vivo estrogenic or anti-estrogenic effects. This mechanism is associated with some of the health promoting effects of legumes.

The inventors are not aware of any commercial soy product on the global market, enriched in glyceollins. Phytoalexin-enriched foods could be defined as any food prepared from plant material that contains higher concentrations or de novo synthesized levels of phytoalexins resulting from elicitor treatment. Elicitor treatments range from biotic elicitors such as microorganisms (Aspergillus sojae, Aspergillus oryzae, and Rhizopus oligosporus), microorganism cell wall extracts, and carbohydrates to abiotic elicitors including UV induction, wounding (e.g. cutting) heavy metal salts (e.g. CuCl₂) and other chemicals such as iodoacetate. As a proof of concept, it has recently been shown on lab scale, that black soybeans germinated under fungal stress with food grade R. oligosporus could technically be utilized for the production of soy milk and soy yogurt containing glyceollins and oxylipins (Feng et al. 2008). Furthermore, germination of black soybean with R. oligosporus for 3 days was sufficient to reduce stacchyose and raffinose (oligosaccharides which cause flatulence) by 92 and 80%, respectively. This research serves as proof of principle that fungus challenged germination of plant seeds can support a niche in food research, to search and develop new functional foods.

Thus, there is still a need for new and improved nutraceuticals and methods for their production. The product shall have a composition with superior relative estrogen receptor binding characteristics.

### DISCLOSURE OF THE INVENTION

Hence, it is a general object of the invention to provide a soybean derived nutraceutical composition with improved composition of beneficial compounds, in particular phytoalexin-enriched foods that will benefit the consumer by providing health-enhancing food choices.

It was another object of the present invention to provide a method for producing such an improved soybean derived nutraceutical composition. Thus, another benefit is that many underutilized crops may be used, such as other varieties of beans, peas or even cereals that may produce phytoalexins and that so far may not have been considered to be health promoting food.

Now, in order to implement these and still further objects of the invention, which will become more readily apparent as the description proceeds, the soybean nutraceutical of the present invention is manifested by the features that it comprises a composition, in particular a composition derivable from soybean, containing prenylated isoflavones and at least one isoflavonoid, said isoflavonoid being selected from one of the chemical classes of isoflavones, coumestans and pterocarpans.

Thus, one aspect of the present invention is that it has surprisingly been found that a special fungus-challenged germination technique of soybeans leads to a composition with a unique profile of known and novel prenylated isoflavones, coumestans and pterocarpans. The term novel as used in connection with prenylated isoflavones, coumestans and pterocarpans means that these compounds may be known per se but have never been observed in soybean before.

In particular the composition of the invention comprises 7 novel prenylated isoflavones, 2 novel glyceollins (IV and VI) and 1 prenylated coumestrol, all of which were never observed in soybeans before.

In a preferred composition, isoflavones, prenylated isoflavones, coumestans and pterocarpans, are comprised simultaneously.

In the composition, the isoflavones comprise daidzein, glycitein and genistein, and their respective glucosidic forms.

In another preferred composition all of these 8 newly formed prenylated isoflavonoides are comprised, i.e. 7 isoflavones substituted with a prenyl chain and one coumestan which is a prenylated coumestrol (coumestan).

The 7 prenylated isoflavones that are not prenylated coumestrol are assumed to belong to the sub-subclass of isoflavones and are A-ring and B-ring prenylated daidzein, A-ring prenylated 2′-hydroxydaidzein, B-ring prenylated glycitein, A-ring prenylated 2′-hydroxygenistein, A-ring and B-ring prenylated genistein.

The inventive composition preferably comprises at least 5% prenylated isoflavones and more preferred also at least 2% prenylated coumestrol of all identified isoflavonoids in the composition.

In a preferred composition, the pterocarpans are selected from glyceollin I, glyceollin II, glyceollin III, glyceollin IV and mixtures thereof. The composition may further comprise and preferably comprises other pterocarpans such as glyceollidins and glycinol being precursors in the biosynthetic pathway of glyceollins.

In yet another preferred composition the pterocarpans are selected from glyceollin I, glyceollin II, glyceollin III, and glyceollin IV and mixtures thereof, much preferred mixtures of glyceollin I, glyceollin II, glyceollin III, and glyceollin IV in a specific ratio of (0.5-2) to (0.5-2) to (0.5-2) to (0.5-2), indicating that all 4 glyceollins are present in similar relative amounts.

Usually the inventive composition comprises glyceollidins, in particular in an amount of at least 3% of the amount of all isoflavonoids identified (see below).

In another inventive composition isoflavones, glyceollins and coumestans, and the prenylated isoflavones as well as precursors of glyceollins such as glyceollidins and glycinol are simultaneously present.

The pterocarpans are usually present in an amount of at least 40% of the amounts of all isoflavonoids identified.

Upon soaking of soybeans, minor changes occur with respect to isoflavonoid composition. The main peaks are allocated to the well known and expected soy isoflavones, including their glucosidic forms. In merely soaked soybeans genistein and its derivatives form the main isoflavones, over 50% of all isoflavonoids identified. This level is typical for soybeans, to be followed by daidzein and its glucosidic derivatives, that make more than 30% of all isoflavonoids identified. Major changes appear upon germination under fungal challenge: (Prenylated) pterocarpans, prenylated isoflavones and (prenylated) coumestans are formed with the unavoidable consequence of reduced relative levels of the soy isoflavones. These isoflavones are known precursors for most of the induced compounds forming the novel soy seedling composition. Therefore, the resulting composition is completely different from other soybean derived isoflavonoid compositions.

Surprisingly, these compositional changes upon fungus challenged germination were consistently far more dramatic in the pilot scale (also termed intermediate scale) experiments compared to lab scale experiments.

While in merely soaked soybeans the largest peaks were found for daidzein (20%), genistein (28%) and genistin (16%), in lab scale germination comprising germination under stress primarily genistin was reduced to <5%, usually about 1-2%. After up-scaling the daidzein was reduced to about 1-3%, genistein to 1-2% and genistin was reduced to less than 1%. Simultaneously, the amount of prenylated isoflavones raised from about 0% in merely soaked soybeans to more than 5% after stressed germination in lab scale to double the amount in pilot scale.

For obtaining such improved composition, soybeans were soaked and germinated (sprouted) while stressed by a fungus in a micromalting system. This induced the formation of a unique and novel ingredient composition for the further preparation of extracts for health supplements and/or medicaments. Some of the compounds already described although not in soybeans are known to be estrogenic and for the novel ingredients the estrogenic effect, was demonstrated by in vitro binding to estrogen receptors alpha and beta.

In more detail, the method for the production of soybean seedlings comprising the composition of the present invention comprises the following steps:

a) soaking the soybeans

b) germinating the soybeans under stress.

The stress is preferably applied by the presence of cultures of fungi, preferably of Rhizopus microsporus such as Rhizopus microsporus oryzae.

Steps a) and b) may be performed in malting systems commonly used in industry for barley malting.

The composition of the present invention may be isolated from the soybean seedlings by adding a third step c) which comprises preparing an extract from the soybean seedlings.

In a preferred embodiment, in step a) the soybeans are soaked for 3-30 h at 10-60° C. with water, more preferred during 16-30 h at 15-25° C.

In another specific embodiment, in step b) the soaked soybeans are germinated prior to applying stress for 0-120 h at 15-40° C., more preferred for at least 6 hours, and most preferred during 24-72 h at 25-35° C.

In yet another preferred method, in step b), the germination of the soybeans continues after inoculation with a fungus, in particular Rhizopus microsporus oryzae. The soybean seedlings can be inoculated after 0-120 h of mere, unstressed germination, preferably after at least 6 hours, most preferred after 24-72 h. The fungus is allowed to grow at 20-40° C. at humidity close to 100% RH (relative humidity) such as 90-100% RH for 48-120 h, more preferred at 25-35° C. at 95-100% r.h. for 66-78 h.

In yet another preferred embodiment soaking and germination are performed in the dark.

This novel composition can be used in a food supplement or medicament, in particular for treating or preventing estrogen related health conditions.

Such estrogenic health conditions are e.g. prostate functioning, symptoms associated with benign prostate hyperplasia, pre-menstrual syndrome or symptoms associated with menopause or post-menopause, in particular menopausal or postmenopausal symptoms comprising hot flushes, vaginal disorders, mood disturbance, fatigue, osteoporosis, incontinence, hormone related cancers (breast, endometrium, prostate). Cosmetic effects on skin, nails and hair are also included.

For these reasons, phytoalexin-enriched foods will benefit the consumer by providing health-enhancing food choices. The disclosed method will also benefit many underutilized crops, such as other varieties of beans, peas or even cereals that may be used to produce phytoalexins that have not been considered to be health promoting food.

### BRIEF DESCRIPTION OF THE DRAWINGS

The invention will be better understood and objects other than those set forth above will become apparent when consideration is given to the following detailed description thereof. Such description makes reference to the annexed drawings, wherein the

FIG. 1 shows a comparison of soybean seedling derived compositions prepared in lab scale and intermediate scale. The UHPLC-chromatograms show that the compositional changes upon fungus challenged germination were more pronounced in the pilot scale experiments compared to lab scale experiments.

FIG. 2 shows a more detailed version of the UHPLC chromatogram of a composition obtained by intermediate scale process indicating the peaks found for known and unknown components, as elements of the composition. The peak numbers refer to the compounds listed in Table 3.

FIG. 3 shows the gradual increase in estrogenicity of extracts during the induction process towards two estrogen receptors in comparison with the activity of estradiol (E2) set as 1.00.

### MODES FOR CARRYING OUT THE INVENTION

The aim of the present invention was to improve the isoflavonoid composition of processed soybeans, aiming for a variety and range of potentially bio-active isoflavonoids in specific compositions. In order to achieve such improved compositions, germination and fungus challenging experiments were performed in order to induce chemical changes in the soybean. Such changes were induced by germinating the soybeans in the presence of a fungus. Several strains were investigated for inducing advantageous compositional changes, i.e. the generation of potentially estrogenic compounds. Besides the altered isoflavonoid composition, wherein the biosynthesis of the known glyceollins shall be conserved and further prenylated isoflavones (with estrogenic activity) shall be formed also an increased total isoflavonoid amount is desired, such as an amount increased by e.g. a factor 1 to 3 which corresponds to the increase seen in recent preliminary experiments.

Different incubation conditions and fungi were tested in several experiments. Germinating soybeans were inoculated with 1 of 4 different strains of fungi under different growth conditions. Among four different strains, Rhizopus microsporus var. oryzae (also referred to merely as Rhizopus microsporus oryzae) was observed to have the most vigorous growth. The highest glyceollins, daidzein and genistein contents were observed in soybeans inoculated with Rhizopus microsporus var. oryzae. In addition, with this fungus the simultaneous formation of compounds was observed, that had never been described before, some not at all and others not in connection with any plant source and in any case not with soybeans.

The lab scale experiments in glass jars were scaled up to kg-scale in a micro-malting system, commonly used in industrial barley malting studies.

### Example 1

### Lab Scale (Small Scale)

The soybeans were surface sterilised by soaking them in a 1% hypochlorite (m/v) solution (5 l/kg beans) under continuous stirring for 1 hour at 20° C. After surface sterilisation, the soybeans were rinsed with sterile demineralised water and then soaked for 4 hours at 40° C. in sterile Milli-Q water. After soaking the beans were germinated in 370 ml glass jars of which the bottom was covered with filter paper humidified with sterile Milli-Q water to prevent the beans from drying out. The jars were loosely closed with a lid to allow air passage and incubated for 4 days at 30° C. in the dark.

For the fungal inoculation of the soybeans, a sporangiospore suspension was used, prepared by scraping off the sporangia from pure slant cultures, e.g. of Rhizopus microsporus var. oryzae grown on malt extract agar (CM59; Oxoid, Basingstoke, UK) for 7 days at 30° C., and suspending them in sterile Milli-Q water with 0.85% NaCl (10⁸ CFU mL⁻¹). After inoculation with the sporangiospore suspension (0.2 ml g⁻¹), the beans were incubated for an additional 4 days at 30° C. in the dark, during which fungal growth as well as further growth of the seedling took place.

### Example 2

### Scale Up to Pilot Scale

The intermediate scale or pilot scale, respectively, germination of soybeans was tested in an Automated Joe White Malting Systems Micro-malting Unit (Perth, Australia). Under controlled conditions, 6.4 kg soybeans were soaked for 20-24 h at 20° C., germinated for 48 h at 30° C. at 100% r.h. (relative humidity) and then inoculated with Rhizopus microsporus var. oryzae (See example 1 for preparation of spore solution; dose was 0.2 ml g⁻¹ of spore solution (10⁸ CFU mL⁻¹)). The experiment was performed in the micro-malting system including a disinfection step prior to soaking, performed in a similar fashion as in example 1. After inoculation, germination continued for 120 h at 30° C. at 100% r.h. After 72 h conditions were adjusted to avoid oversaturation of circulating air. The seedlings were collected, freeze-dried and extracted.

### Example 3

### Analytics

Equipment and Procedure:

Fresh soybeans, soaked soybeans and fungus-challenged germinated soybeans were freeze-dried and then milled to yield a powder with a particle size smaller than 1 mm. The powder was defatted by hexane extraction for 30 min in a sonication bath at 30° C. (0.04 g powder/ml hexane). After defatting, the flavonoids were extracted with absolute EtOH (0.04 g powder/ml EtOH) by a two-step sequential extraction of the defatted powder with each solvent for 30 min in a sonication bath at 30° C. The extracts were centrifuged at 2500 g for 15 min. The supernatant was collected and the solvent evaporated resulting in dried extracts. The dried extracts were resolubilised in methanol (MeOH) to yield a stock concentration of 10 mg mL⁻¹ and stored at −20° C. All samples were thawed and centrifuged before analysis.

Samples were analysed on a UHPLC (ultra high pressure liquid chromatography) system equipped with pump, auto-sampler and PDA (photodiode array) detector. Samples (1 μl) were injected on a Waters Acquity UPLC BEH shield RP18 column with a Waters Acquity UPLC shield RP18 Vanguard pre-column. Water acidified with 0.1% (v/v) acetic acid, eluent A, and acetonitrile (ACN) acidified with 0.1% (v/v) acetic acid, eluent B, were used as eluents. The flow rate was 300 μL min⁻¹, the column oven temperature controlled at 25° C., and the PDA detector was set to measure at a range of 200-400 nm. The following elution profile was used: 0-2 min, linear gradient from 10%-25% (v/v) B; 2-9 min, linear gradient from 25%-50% (v/v) B; 9-12 min, isocratic on 50% B; 12-22 min, linear gradient from 50%-100% (v/v) B; 22-25 min, isocratic on 100% B; 25-27 min, linear gradient from 100%-10% (v/v) B; 27-29 min, isocratic on 10% (v/v) B. Mass spectrometric data were obtained by analysing samples on a Thermo Scientific LTQ-XL equipped with an ESI-MS probe coupled to the RP-UHPLC. Helium was used as sheath gas and nitrogen as auxiliary gas. Data were collected over an m/z-range of 150-1500. Data dependent MSⁿ analysis was performed with a normalised collision energy of 35%. The MSⁿ fragmentation was always performed on the most intense daughter ion in the MSⁿ⁻¹ spectrum. Most settings were optimised using “tune plus” via automatic tuning.

The system was tuned with genistein in both positive ionisation mode (PI mode) and negative ionisation mode (NI mode). In the NI mode, the ion transfer tube temperature was 350° C. and the source voltage 4.8 kV. Both in the PI and NI mode, the ion transfer tube temperature was 350° C. and the source voltage 4.8 kV. Data acquisition and reprocessing were done with Xcalibur 2.0.7. Because analytical reference HPLC-standards were unavailable for most of the compounds identified, quantification of all compounds was done as daidzein equivalent in mg/g, using isolated daidzein (purity min. 98%) purchased from Wako Chemicals (Neuss, Germany). The composition are therefore expressed is % present of the total isoflavonoids present and identified.

In Vitro Activity Testing:

A yeast based assay was used to demonstrate estrogenic acitivity of extracts. The principle of this bio-assay is described in (Bovee et al. 2004a). Samples were solubilised in DMSO (10 mg/ml) that was used as a stock solution for further dilution. A series of concentrations was pipetted (2 μl) in a 96-micro well (MW) plate. The assay was performed as described in (Bovee et al. 2004b).

Outcome/Results:

Small Scale (Lab Scale) Experiments:

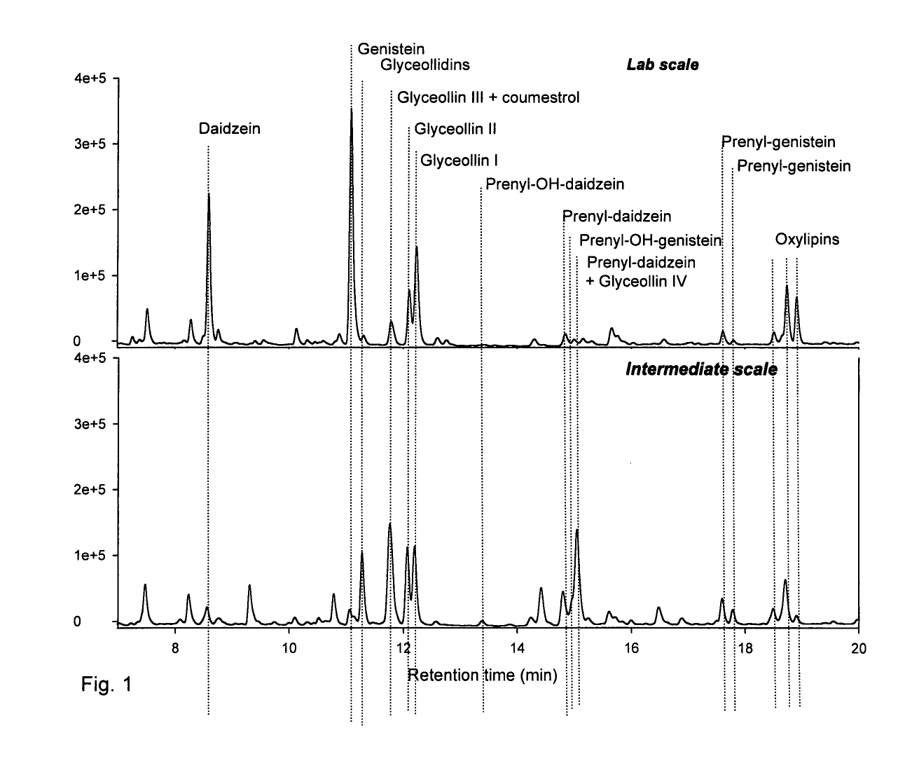

The UHPLC-UV profiles of the EtOH extracts of unsoaked beans, soaked beans and fungi-challenged germinated beans show the changes in isoflavonoid composition taking place upon soaking, followed by germination and fungi-challenged germination. A total of 30 peaks were tentatively assigned in all 3 UHPLC profiles (see FIG. 1 and correlation of retention time to compound in Table 3). The UHPLC-UV profile of unsoaked and soaked soybeans was characterised by the presence of the main soy isoflavones genistein, daidzein, glycitein and their glucoside derivatives. Of the isoflavone aglycones, genistein and daidzein were most abundant.

Upon germination followed by fungi-challenged germination, the glucoside conjugated isoflavones decreased to minor peaks, whereas daidzein, glycitein, genistein and malonyl genistin became the dominating peaks in the UV-profile. In addition, several new peaks appeared in the chromatogram (see FIG. 1, Table 4).

The first cluster of peaks in FIG. 1 eluted right after genistein and were tentatively assigned as, a co-eluting peak for glyceollidin I+II and coumestrol, followed by glyceollins I, II, III, IV and VI. Glyceollins and their precursors glyceollidins are prenylated pterocarpans that are derived from glycinol, a non-prenylated pterocarpan that was also found in the UV-profile of fungi-challenged soybean seedlings. In addition, a group of oxylipins was found to elute in the second half of the chromatogram. These peaks were tentatively assigned as oxooctadecadienoic acids (KODEs) (Feng et al. 2007), but are not of interest for this invention.

The presence of several prenylated isoflavonoids, such as the pterocarpans glyceollins I, II, III, IV and VI and glyceollidins I/II, makes the extracts of fungi-challenged soybean seedlings an interesting source to screen for possible other prenylated flavonoids. Several new prenylated isoflavonoids have been induced that had never been observed as constituents in soybean products.

Intermediate Scale (Pilot Scale) Experiment:

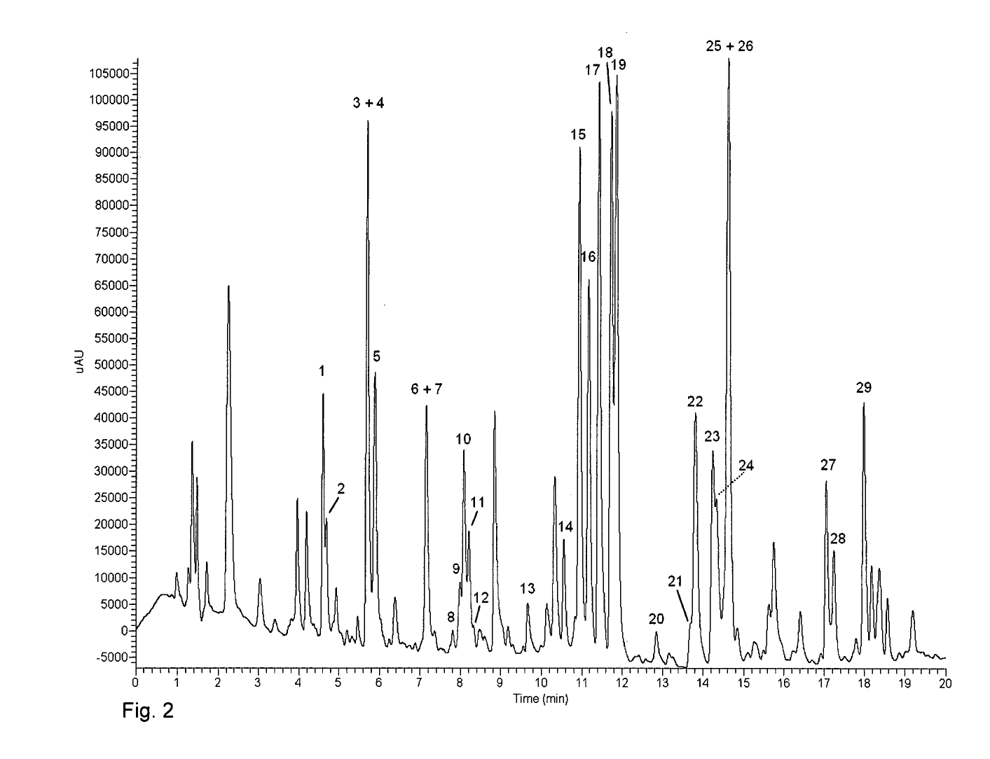

As can be seen in the UHPLC-UV profile, upscaling caused a further increase in content of the novel compounds (eluting between 14 and 18 min) upon induction, leading to a composition comprising 4 main groups of compounds: “traditional” soy isoflavones, coumestans including prenylated coumestrol, pterocarpans (glycinol, glyceollins and glyceollidins), and novel prenylated isoflavones (see FIGS. 1 and 2 (the correlation between the peak number and the compound can be found in Table 3), Tables 4 and 5). Such a composition has not been described before.

TABLE 3

| No | Rt UV | Tentative ID |
|---|---|---|
| 1 | 4.60 | Daidzin |
| 2 | 4.66 | Glycitin |
| 3 | 5.69 | Genistin |
| 4 | 5.70 | Glycinol |
| 5 | 5.89 | Mal-Daidzin |
| 6 | 7.15 | Mal-Genistin |
| 7 | 7.16 | 2′-OH-daidzein |
| 8 | 7.81 | Biochanin A |
| 9 | 7.99 | Glycitein |
| 10 | 8.08 | Glyceofuran |
| 11 | 8.21 | Daidzein |
| 12 | 8.32 | 2′-OH-genistein |
| 13 | 9.67 | Naringenin |
| 14 | 10.56 | Genistein |
| 15 | 10.94 | Glyceollidin I/II |
| 16 | 11.17 | Coumestrol |
| 17 | 11.42 | Glyceollin III |
| 18 | 11.73 | Glyceollin II |
| 19 | 11.85 | Glyceollin I |
| 20 | 12.85 | Ap-2′OH-daidzein |
| | | [prenyl-OH- |
| | | daidzein] |
| 21 | 13.70 | Bp-glycitein [prenyl- |
| | | glycitein (b)] |
| 22 | 13.82 | Glyceollin VI (Clan- |
| | | destacarpin) |
| 23 | 14.24 | Ap-daidzein [prenyl- |
| | | daidzein (a)] |
| 24 | 14.33 | Ap-2′OH-genistein |
| | | [prenyl-OH- |
| | | genistein] |
| 25 | 14.52 | Bp-daidzein [prenyl- |
| | | daidzein (b)] |
| 26 | 14.62 | Glyceollin IV |
| 27 | 17.06 | Ap-Genistein [prenyl- |
| | | genistein (a)] |
| 28 | 17.25 | Bp-Genistein [prenyl- |
| | | genistein (b)] |
| 29 | 17.99 | Phaseol [prenyl- |
| | | coumestrol] |

TABLE 4

| | | Soaked Soy beans | | Lab scale fermentation | | Micro-malting scale (pilot) | |
|---|---|---|---|---|---|---|---|
| | | | abs. est. content | | abs. est. content | | abs. est. content |
| | | rel. content as | as daidzein eq. | rel. content as | as daidzein eq. | rel. content as | as daidzein eq. |
| Class | Compound | Daidzein eq. | (mg/100 mg extract) | Daidzein eq. | (mg/100 mg extract) | Daidzein eq. | (mg/100 mg extract) |
| Isoflavones | Daidzein | 20.0% | 0.5 | 16.0% | 2.1 | 2.0% | 0.3 |
| | Daidzin | 12.0% | 0.3 | 1.5% | 0.2 | 1.3% | 0.2 |
| | Malonyl daidzin | 12.0% | 0.3 | 2.3% | 0.3 | 5.4% | 0.8 |
| | Acetyl daidzin | | <0.1 | | <0.1 | | <0.1 |
| | Glycitein | | <0.1 | 3.1% | 0.4 | 3.4% | 0.5 |
| | Glycitin | 4.0% | 0.1 | 0.8% | 0.1 | 0.7% | 0.1 |
| | Malonyl glycitin | | <0.1 | | <0.1 | | <0.1 |
| | Acetyl glycitin | | <0.1 | | <0.1 | | <0.1 |
| | Genistein | 28.0% | 0.7 | 27.5% | 3.6 | 1.3% | 0.2 |
| | Genistin | 16.0% | 0.4 | 2.3% | 0.3 | 0.7% | 0.1 |
| | Malonyl genistin | 8.0% | 0.2 | 3.8% | 0.5 | 4.7% | 0.7 |
| | Acetyl genistin | | <0.1 | | <0.1 | | <0.1 |
| | OH-genistein | | 0 | 1.5% | 0.2 | 1.3% | 0.2 |
| | Biochanin A | | <0.1 | | <0.1 | 0.7% | 0.1 |
| Coumestans | Coumestrol | 0.0% | 0 | 0.8% | 0.1 | 6.0% | 0.9 |
| | Prenyl coumestrol | 0.0% | 0 | 7.6% | 1.0 | 6.0% | 0.9 |
| Pterocarpans | Glyceollin I | 0.0% | 0 | 13.0% | 1.7 | 8.7% | 1.3 |
| | Glyceollin II | 0.0% | 0 | 6.1% | 0.8 | 8.1% | 1.2 |
| | Glyceollin III | 0.0% | 0 | 2.3% | 0.3 | 9.4% | 1.4 |
| | Glyceollin IV | 0.0% | 0 | | <0.1 | 11.4% | 1.7 |
| | Glyceofuran | 0.0% | 0 | | <0.1 | 3.4% | 0.5 |
| | Glycinol | 0.0% | 0 | 3.8% | 0.5 | 4.7% | 0.7 |
| | Glyceollidin I/II | 0.0% | 0 | 1.5% | 0.2 | 7.4% | 1.1 |
| Prenylated | Prenyl daidzein (a) | 0.0% | 0 | 1.5% | 0.2 | 4.0% | 0.6 |
| isoflavones | Prenyl daidzein (b) | 0.0% | 0 | 0.8% | 0.1 | 1.3% | 0.2 |
| | Prenyl-glycitein | 0.0% | 0 | 0.8% | 0.1 | 1.3% | 0.2 |
| | Prenyl-OH-daidzein | 0.0% | 0 | | <0.1 | 0.7% | 0.1 |
| | Prenyl-OH-genistein | 0.0% | 0 | 0.8% | 0.1 | 2.0% | 0.3 |
| | Prenyl genistein (a) | 0.0% | 0 | 1.5% | 0.2 | 2.7% | 0.4 |
| | Prenyl genistein (b) | 0.0% | 0 | 0.8% | 0.1 | 1.3% | 0.2 |
| | | Sum = 100 | 2.5 | Sum = 100 | 13.1 | Sum = 100 | 14.9 |

TABLE 5

| | | Intermediate scale | | Relative | |
|---|---|---|---|---|---|
| | | Retention | rel. | content per | Ranges of |
| Sub- | | time | content as | chemical sub- | relative |
| subclass | Compound | (min)* | Daidzein eq. | subclass | amounts |
| Isoflavones | Daidzein | 8.21 | 2.0% | 21.5% | 15-30% |
| | Daidzin | 4.60 | 1.3% | | |
| | Malonyl daidzin | 5.89 | 5.4% | | |
| | Acetyl daidzin | | | | |
| | Glycitein | 7.99 | 3.4% | | |
| | Glycitin | 4.66 | 0.7% | | |
| | Malonyl glycitin | | | | |
| | Acetyl glycitin | | | | |
| | Genistein | 10.56 | 1.3% | | |
| | Genistin | 5.69 | 0.7% | | |
| | Malonyl genistin | 7.15 | 4.7% | | |
| | Acetyl genistin | | | | |
| | OH-genistein | 8.32 | 1.3% | | |
| | Biochanin A | 7.81 | 0.7% | | |
| Coumestans | Coumestrol | 11.17 | 6.0% | 12% | 5-20% |
| | Prenyl coumestrol | 17.99 | 6.0% | | such as |
| | | | | | 7-20% |
| Pterocarpans | Glyceollin I | 11.85 | 8.7% | 53.1% | 40-60% |
| | Glyceollin II | 11.73 | 8.1% | | |
| | Glyceollin III | 11.42 | 9.4% | | |
| | Glyceollin IV | 14.62 | 11.4% | | |
| | Glyceofuran | 8.08 | 3.4% | | |
| | Glycinol | 5.70 | 4.7% | | |
| | Glyceollidin I/II | 10.94 | 7.4% | | |
| Prenylated | Prenyl daidzein (a) | 14.24 | 4.0 | 13.3% | 5-20% |
| isoflavones | Prenyl daidzein (b) | 14.52 | 1.3 | | |
| | Prenyl-glycitein | 13.70 | 1.3 | | |
| | Prenyl-OH-daidzein | 12.85 | 0.7 | | |
| | Prenyl-OH-genistein | 14.33 | 2.0 | | |
| | Prenyl genistein (a) | 17.06 | 2.7 | | |
| | Prenyl genistein (b) | 17.25 | 1.3 | | |
| Sum | | | | 100% | 100% |

*retention times may fluctuate over time, however sequence of peaks is stable.

This composition was tested in the yeast-based bioactivity assay.

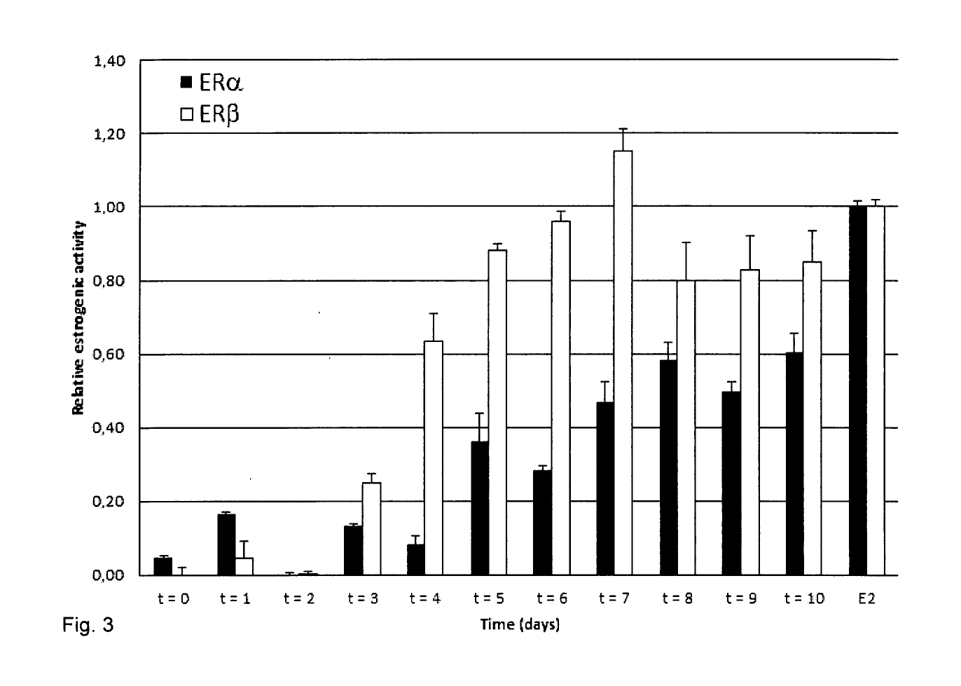

With a final extract concentration in the assay of 10 μg/ml, a clear increase in estrogenic activity for the ERα in time was observed. The same increasing trend was observed for the ERβ in time, when measured at a final concentration of 1 μg/ml (see FIG. 3). FIG. 3 shows the gradual increase in estrogenicity of the extracts during the induction process towards both estrogen receptors.

### CONCLUSION

It has been found that upon soaking of fresh soybeans the peaks for the compounds daidzein, genistein and genistin in the UV profile of an LC-chromatogram are the most dominant peaks. After soaking followed by germination and fungal challenged germination of soybeans in lab scale, daidzein and genistein still were the dominating peaks in the UV-profile compared to the results of non-germinated and non stressed soybeans, however new peaks appeared in the chromatogram that could be assigned as representatives of coumestans, pterocarpans, isoflavones and prenylated isoflavones. Ten prenylated isoflavonoids, new to soybean products, could be induced. Surprisingly the induction of these novel compounds could be increased by upscaling the germination. This upscaling surprisingly led to a further, novel and unexpected chemical composition with enhanced levels of the not yet observed compounds. Of the 8 new prenylated isoflavonoids, 7 belong to the sub-subclass of isoflavones and were tentatively assigned as A-ring (a) and B-ring (b) prenylated daidzein, A-ring prenylated 2′-hydroxydaidzein, B-ring prenylated glycitein, A-ring prenylated 2′-hydroxygenistein, A-ring and B-ring prenylated genistein. In addition, a further prenylated isoflavonoid was tentatively assigned as prenylated coumestrol (coumestan).

Presently only either processed whole soybean, fermented or sprouted soy products are on the market as foodstuff, or extracts therefrom are formulated in food supplements. These products mostly contain daidzein, glycitein and genistein and their glucoside forms as active ingredients.

The improved composition of the soybeans treated according to the present invention with the broader spectrum of bioactive health ingredients qualifies them as health food, supplements and/or for the production of medicaments with much higher health supporting activity compared to products made out of untreated soybeans.

A further benefit is that due to the higher content of healthy components, lower dosage recommendations are needed leading to a more comfortable intake for the end users.

A further advantage is the possibility to produce fungus-challenged germinated soybean seedlings in large, i.e. industrial production scale.

While there are shown and described presently preferred embodiments of the invention, it is to be distinctly understood that the invention is not limited thereto but may be otherwise variously embodied and practiced within the scope of the following claims.

### LITERATURE

Ahn E M, Nakamura N, Akao T, Nishihara T & Hattori M. 2004. Estrogenic and antiestrogenic activities of the roots of Moghania philippinensis and their constituents. Biological and pharmaceutical bulletin 24(4):548-553
Booth N L, Overk C R, Yao P, Burdette J E, Nikolic D, Chen S N, Bolton J L, Van Breemen R B, Pauli G F & Farnsworth N R. 2006. The chemical and biologic profile of a red clover (Trifolium pratense L.) phase II clinical extract. Journal of Alternative and Complementary Medicine 12(2):133-139.
Boué S M, Cleveland T E, Carter-Wientjes C, Shih B Y, Bhatnagar D, McLachlan J M & Burow M E. 2009. Phytoalexin-Enriched Functional Foods. Journal of Agricultural and Food Chemistry 57(7):2614-2622.
Bovee T F H, Helsdingen R J R, Koks P D, Kuiper H A, Hoogenboom RLAP & Keijer J. 2004a. Development of a rapid yeast estrogen bioassay, based on the expression of green fluorescent protein. Gene 325(1-2):187-200.
Bovee T F H, Helsdingen R J R, Rietjens I M C M, Keijer J & Hoogenboom R L A P. 2004b. Rapid yeast estrogen bioassays stably expressing human estrogen receptors
a and β, and green fluorescent protein: A comparison of different compounds with both receptor types. Journal of Steroid Biochemistry and Molecular Biology 91(3):99-109.
Burow M E, Boué S M, Collins-Burow B M, Melnik L I, Duong B N, Carter-Wientjes C H, Li S, Wiese T E, Cleveland T E & McLachlan J A. 2001. Phytochemical glyceollins, isolated from soy, mediate antihormonal effects through estrogen receptor α and β. Journal of Clinical Endocrinology and Metabolism 86(4):1750-1758.
Dakora F D & Phillips D A. 1996. Diverse functions of isoflavonoids in legumes transcend anti-microbial definitions of phytoalexins. Physiological and Molecular Plant Pathology 49(1):1-20.
Feng S, Chin L S, Yuan K L & Huang D. 2007. Fungal-stressed germination of black soybeans leads to generation of oxooctadecadienoic acids in addition to glyceollins. Journal of Agricultural and Food Chemistry 55(21):8589-8595.
Feng S, Saw C L, Lee Y K & Huang D. 2008. Novel process of fermenting black soybean [Glycine max (L.) Merrill] yogurt with dramatically reduced flatulence-causing oligosaccharides but enriched soy phytoalexins. Journal of Agricultural and Food Chemistry 56(21):10078-10084.
Graham T L & Graham M Y. 1991. Glyceollin elicitors induce major but distinctly different shifts in isoflavonoid metabolism in proximal and distal soybean cell-populations. Molecular Plant-Microbe Interactions 4(1):60-68.
Graham T L, Kim J E & Graham M Y. 1990. Role of constitutive isoflavone conjugates in the accumulation of glyceollin in soybean infected with Phytophthora megasperma Molecular Plant-Microbe Interactions 3(3):157-166.
Han D H, Denison M S, Tachibana H & Yamada K. 2002. Relationship between estrogen receptor-binding and estrogenic activities of environmental estrogens and suppression by flavonoids. Bioscience, Biotechnology and Biochemistry 66(7):1479-1487.
Keen N T, Lyne R L & Hymowitz T. 1986. Phytoalexin Production as a Chemosystematic Parameter Within the Genus Glycine. Biochemical Systematics and Ecology 14(5):481-486.
Kretzschmar G, Zierau O, Wober J, Tischer S, Metz P & Vollmer G. 2010. Prenylation has a compound specific effect on the estrogenicity of naringenin and genistein. Journal of Steroid Biochemistry and Molecular Biology 118(1-2):1-6.
Mazur W M, Duke J A, Wahala K, Rasku S & Adlercreutz H. 1998. Isoflavonoids and lignans in legumes: Nutritional and health aspects in humans. Journal of Nutritional Biochemistry 9(4):193-200.
Messina M & Bennink M. 1998. Soyfoods, isoflavones and risk of colonic cancer: A review of the in vitro and in vivo data. Bailliere's Clinical Endocrinology and Metabolism 12(4):707-728.
Okamoto Y, Suzuki A, Ueda K, Ito C, Itoigawa M, Furukawa H, Nishihara T & Kojima N. 2006. Anti-estrogenic activity of prenylated isoflavones from Millettia pachycarpa: Implications for pharmacophores and unique mechanisms. Journal of Health Science 52(2):186-191.
Paxton J D. 1991. Biosynthesis and accumulation of legume phytoalexins. In: Sharma, R. P. & Salunkhe, D. K., editors. Mycotoxins and Phytoalexins. Boca Raton, Fla.: CRC press. p. 485-500.
Price K R & Fenwick G R. 1985. Naturally occurring oestrogens in foods—A review. Food Additives and Contaminants 2(2):73-106.
Salvo V A, Boué S M, Fonseca J P, Elliott S, Corbitt C, Collins-Burow B M, Curiel T J, Srivastav S K, Shih B Y, Carter-Wientjes C, Wood C E, Erhardt P W, Beckman B S, McLachlan J A, Cleveland T E & Burow M E. 2006. Antiestrogenic glyceollins suppress human breast and ovarian carcinoma tumorigenesis. Clinical Cancer Research 12(23):7159-7164.
Sun Y C, Tae Y H, Ji Y A, Sung R K, Kyung S K, In K H & Kim S. 2008. Estrogenic activities of isoflavones and flavones and their structure-activity relationships. Planta Medica 74(1):25-32.
Wood C E, Clarkson T B, Appt S E, Franke A A, Boue S M, Burow M E, McCoy T & Cline J M. 2006. Effects of soybean glyceollins and estradiol in postmenopausal female monkeys. Nutrition and Cancer 56(1):74-81.
Zimmermann M C, Tilghman S L, Boué S M, Salvo V A, Elliott S, Williams K Y, Skripnikova E V, Ashe H, Payton-Stewart F, Vanhoy-Rhodes L, Fonseca J P, Corbitt C, Collins-Burow B M, Howell M H, Lacey M, Shih B Y, Carter-Wientjes C, Cleveland T E, McLachlan J A, Wiese T E, Beckman B S & Burow M E. 2010. Glyceollin I, a novel antiestrogenic phytoalexin isolated from activated soy. Journal of Pharmacology and Experimental Therapeutics 332(1):35-45.

1. A composition, in particular a composition derivable from soybean, containing prenylated isoflavones and at least one isoflavonoid, said isoflavonoid being selected from one of the chemical classes of isoflavones, coumestans and pterocarpans.

2. The composition of claim 1 containing at least three isoflavonoids, at least one of each of the chemical classes of isoflavones, coumestans and pterocarpans.

3. The composition of claim 1, comprising at least 5% prenylated isoflavones of the total amount of isoflavonoids present.

4. The composition of claim 1, wherein the isoflavonoids comprise coumestans in an amount of at least 4% of the total isoflavonoids present and wherein the coumestans comprise prenylated coumestrol.

5. The composition of claim 1, wherein the prenylated isoflavonoids comprise isoflavones selected from the group consisting of A-ring and B-ring prenylated daidzein, A-ring prenylated 2′-hydroxydaidzein, B-ring prenylated glycitein, A-ring prenylated 2′-hydroxygenistein, A-ring and B-ring prenylated genistein and mixtures thereof.

6. The composition of claim 1, wherein the isoflavonoids comprise pterocarpans in an amount of at least 40% of the total amount of isoflavonoids.

7. The composition of claim 1, wherein the pterocarpans are selected from glyceollin I, glyceollin II, glyceollin III and glyceollin IV.

8. The composition of claim 1 wherein glyceollin I, glyceollin II, glyceollin III and glyceollin IV are present in a specific ratio of (0.5-2) to (0.5-2) to (0.5-2) to (0.5-2).

9. The composition of claim 1 further comprising at most 5% genistin, preferably at most 2% genistin.

10. A method for the production of soybean seedlings comprising the composition of claim 1, comprising the steps:
a) soaking the soybeans; and
b) germinating the soybeans, at least partially under stress.

11. The method of claim 10, wherein steps a) and b) are performed in a malting system commonly used for barley malting.

12. The method of claim 10, wherein step a) is performed by soaking the soybeans for 3-30 h at 10-60° C. with water, more preferred during 16-30 h at 15-25° C.

13. The method of claim 10, wherein step b) is performed by germinating the soaked soybeans prior to applying stress for 0-120 h at 15-40° C., more preferred for at least 6 hours, most preferred during 24-72 h at 25-35° C.

14. The method of claim 10, wherein stress is applied in step b) by continued germination combined with incubation of the soybeans after inoculation with a fungus, in particular Rhizopus microsporus var. oryzae.

15. The method of claim 10, wherein inoculation is performed after 0-120 h of germination of the soaked soybean, preferably after at least 6 hours, most preferred after 24-72 h.

16. The method of claim 10, wherein the incubation is performed at 20-40° C. at 90-100% RH for 48-120 h, more preferred at 25-35° C. at 95-100% RH for 66-78 h.

17. The method of claim 10, wherein the soaking and germination are performed in the dark.

18. The method of claim 10, wherein after step b) the soybean seedlings are subjected to an extraction step c) for preparing an extract having the composition claim 1.

19. Use of the composition of claim 1 in or as a food supplement or in cosmetics for use on skin, nails and hair.

20. The composition of claim 1 of for use as medicament, in particular in the prevention and treatment of estrogen-related health conditions, in particular pre-menstrual syndrome or symptoms associated with menopause or post-menopause, preferably menopausal or postmenopausal symptoms comprising hot flushes, vaginal disorders, incontinence, mood disturbance, fatigue or osteoporosis; prostate functioning and symptoms associated with benign prostate hyperplasia; hormone related cancers (breast, endometrium, prostate).

21. Use of Rhizopus microsporus var. oryzae in the production of fermented soybean seedlings and compositions extracted thereof.
